(19) United States
(12) Patent Application Publication
(10) Pub. No.: US 2009/0018355 A1
(43) Pub. Date: Jan. 15, 2009

(54) Process for Crystallizing an Epoxy Compound

(75) Inventor: Shinya Tanaka, Toyono-gun (JP)

Correspondence Address: WENDEROTH, LIND & PONACK, L.L.P., 2033 K STREET N. W., SUITE 800, WASHINGTON, DC, 20006-1021, US

(73) Assignee: Sumitomo Chemical Company, Ltd.

(21) Appl. No.: 12/224,958

(22) PCT Filed: Mar. 9, 2007
(86) PCT No.: PCT/JP2007/055312

§ 371 Date: Sep. 10, 2008

(30) Foreign Application Priority Data
Mar. 15, 2006 (JP) 2006-070392

(51) Int. Cl. C07D 301/32

(52) U.S. Cl. 549/541

(57) ABSTRACT

A process for crystallizing an epoxy compound, which comprises the steps of:

(A) cooling a solution containing a crude product of an epoxy compound, which contains as a main component the epoxy compound represented by the formula (1):

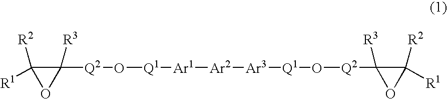

wherein Ar¹, Ar² and Ar³ are the same or different and each denotes any one of divalent groups represented by the following formulas:

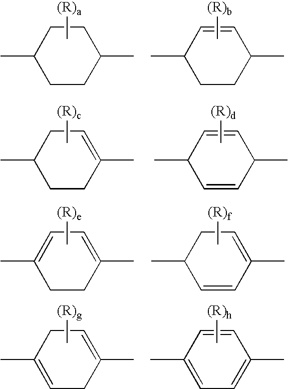

to an internal temperature T¹° C. to precipitate crystals of the epoxy compound represented by the formula (1), thus obtaining a suspension;
(B) heating the suspension obtained in the step (A) to a temperature T²° C. which satisfies the formula (I)

T¹<T²<T (I)

wherein T denotes a saturation temperature of the epoxy compound in the solution containing a crude product of the epoxy compound used in the step (A); and
(C) cooling the suspension heated to the temperature T²° C. in the step (B) to a temperature T³° C. which satisfies the following formula (II).

T³<T¹ (II)

### TECHNICAL FIELD

The present invention relates to a process for crystallizing an epoxy compound.

### BACKGROUND ART

An epoxy compound represented by the formula (1):

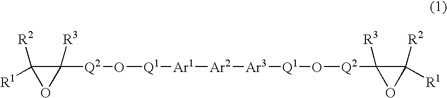

wherein,

Q¹ denotes a single bond or a straight-chain alkylene group having 1 to 9 carbon atoms, in which at least one methylene group composing the straight-chain alkylene group is optionally substituted with an alkyl group having 1 to 8 carbon atoms and —O— or —N(R⁴)— is optionally inserted between the methylene groups, in which R⁴ denotes hydrogen atom or an alkyl group having 1 to 8 carbon atoms;

Q² denotes a straight-chain alkylene group having 1 to 8 carbon atoms, in which at least one methylene group composing the straight-chain alkylene group is optionally substituted with an alkyl group having 1 to 8 carbon atoms;

Ar¹, Ar² and Ar³ are the same or different and each denotes any one of divalent groups represented by the following formulas:

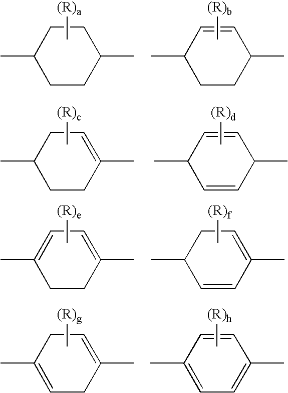

in which R denotes an alkyl group having 1 to 8 carbon atoms, a denotes an integer of 0 to 8, b, e and g denotes an integer of 0 to 6, c denotes an integer of 0 to 7, d and h denote an integer of 0 to 4, and f denotes an integer of 0 to 5, and when more than one R exists in said divalent group, all of R may be the same group or different groups; and

R¹, R² and R³ are the same or different and each denotes hydrogen atom or an alkyl group having 1 to 8 carbon atoms; had a lower melt temperature and can be melt-blended with a curing agent at a curing temperature or lower, as described in EP 1698625 A1. A cured epoxy resin obtained by curing the epoxy compound with the use of a curing agent not only exhibits liquid crystallinity, but also has a high thermal conductivity, so that it is useful as an insulating material which requires a high heat dissipation capacity, such as a printed wiring board.

EP 1698625 A1 discloses a method comprising removing an unreacted epihalohydrin from a reaction mixture containing an epoxy compound, adding a solvent to obtain a solution, and cooling the solution to precipitate crystals of the epoxy compound.

However, a further industrial improvement has been required from the point of view of purity and filterability of the obtained epoxy compound.

### DISCLOSURE OF INVENTION

The present invention provides:

<1> a process for crystallizing an epoxy compound, which comprises the steps of:
(A) cooling a solution containing a crude product of an epoxy compound, which contains as a main component an epoxy compound represented by the formula (1):

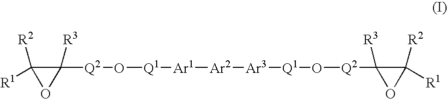

wherein

Ar¹, Ar² and Ar³ are the same or different and each denotes any one of divalent groups represented by the following formulas:

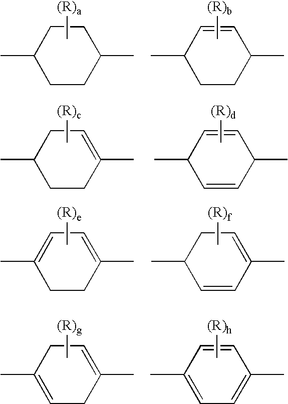

in which R denotes an alkyl group having 1 to 8 carbon atoms, a denotes an integer of 0 to 8, b, e and g denote an integer of 0 to 6, c denotes an integer of 0 to 7, d and h denote an integer of 0 to 4, and f denotes an integer of 0 to 5, and when more than one R exists in said divalent group, all of R may be the same group or different groups;

R¹, R² and R³ are the same or different and each denotes hydrogen atom or an alkyl group having 1 to 8 carbon atoms;

Q¹ denotes a single bond or a straight-chain alkylene group having 1 to 9 carbon atoms, in which at least one methylene group composing the straight-chain alkylene group is optionally substituted with an alkyl group having 1 to 8 carbon atoms and —O— or —N(R⁴)— is optionally inserted between the methylene groups, in which R⁴ denotes hydrogen atom or an alkyl group having 1 to 8 carbon atoms; and

Q² denotes a straight-chain alkylene group having 1 to 8 carbon atoms, in which at least one methylene group composing the straight-chain alkylene group is optionally substituted with an alkyl group having 1 to 8 carbon atoms, to an internal temperature T¹° C. to precipitate crystals of the epoxy compound represented by the formula (1), thus obtaining a suspension;

(B) heating the suspension obtained in the above-described step (A);
to a temperature T²° C. which satisfies the formula (I):

T¹<T²<T (I)

wherein T denotes a saturation temperature of the epoxy compound in the solution containing a crude product of the epoxy compound used in the above-described step (A); and
(C) cooling the suspension heated to the temperature T²° C. in the above-described step (B) to a temperature T³° C. which satisfies the following formula (II):

T³<T¹ (II);

<2> the process for crystallizing an epoxy compound according to <1>, wherein a solvent of the solution containing a crude product of the epoxy compound is at least one selected from the group consisting of an aliphatic ketone solvent, an ether solvent and an aromatic hydrocarbon solvent;
<3> the process for crystallizing an epoxy compound according to <1> or <2>, wherein the temperature T° C. is from 30 to 60° C.;
<4> the process for crystallizing an epoxy compound any one of <1> to <3>, wherein the concentration of the epoxy compound in the solution containing a crude product of the epoxy compound is from 10 to 40% by weight and the temperature T¹ is a temperature which satisfies the following formula (III):

T¹<T−5 (III);

<5> The process for crystallizing an epoxy compound according to any one of <1> to <4>, wherein the temperature T² is a temperature which satisfies the following formula (IV):

T−5<T²<T (IV);

<6> the process for crystallizing an epoxy compound according to any one of <1> to <5>, wherein the temperature T³ is a temperature which satisfies the following formula (V):

−10<T³<T−5 (V);

<7> the process for crystallizing an epoxy compound according to any one of <1> to <6>, wherein the temperature T¹ is from 10 to 30° C.;
<8> the process for crystallizing an epoxy compound according to any one of <1> to <7>, wherein the temperature T² is from 30 to 60° C.;
<9> the process for crystallizing an epoxy compound according to any one of <1> to <8>, wherein the temperature T³ is from −10 to 20° C.;
<10> the process for crystallizing an epoxy compound according to any one of <1> to <9>, wherein Ar¹ and Ar³ independently denote a group represented by the following formula:

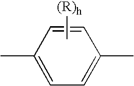

wherein R and h are as defined above;
<11> the process for crystallizing an epoxy compound according to anyone of <1> to <10>, wherein Ar¹ and Ar³ independently denote 1,4-phenylene group, 3-methyl-1,4-phenylene group or 3-isopropyl-1,4-phenylene group;
<12> the process for crystallizing an epoxy compound according to any one of <1> to <11>, wherein Ar² is a group represented by the following formula:

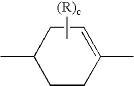

wherein R and c are as defined above;
<13> the process for crystallizing an epoxy compound according to any one of <1> to <12>, wherein Ar² is 1-cyclohexene-1,4-diyl group;
<14> the process for crystallizing an epoxy compound according to any one of <1> to <9>, wherein Ar¹ and Ar³ independently denote a group represented by the following formula:

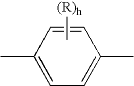

wherein R and h are as defined above, and Ar² denotes a group represented by the following formula:

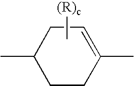

wherein R and c are as defined above; and
<15> the process for crystallizing an epoxy compound according to any one of <1> to <9>, wherein Ar¹ and Ar³ independently denote 1,4-phenylene group, 3-methyl-1,4-phenylene group or 3-isopropyl-1,4-phenylene group, and Ar² denotes 1-cyclohexene-1,4-diyl group.

### BEST MODE FOR CARRYING OUT THE INVENTION

First, the step (A) will be described.

In the formula of the epoxy compound represented by the formula (1):

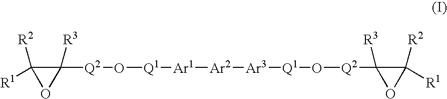

(hereinafter abbreviated to the epoxy compound (1)),

Ar¹, Ar² and Ar³ are the same or different and each denotes any one of divalent groups represented by the following formulas:

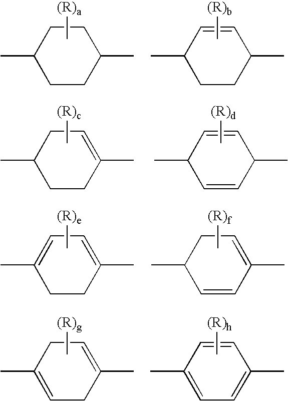

in which R denotes alkyl group having 1 to 8 carbon atoms, a denotes an integer of 0 to 8, b, e and g denote an integer of 0 to 6, c denotes an integer of 0 to 7, d and h denote an integer of 0 to 4, and f denotes an integer of 0 to 5.

Examples of the alkyl group having 1 to 8 carbon atoms include straight-chain, branched chain or cyclic alkyl groups such as methyl, ethyl, n-propyl, isopropyl, n-butyl, isobutyl, sec-butyl, tert-butyl, n-pentyl, tert-pentyl, n-hexyl, 1-methylpentyl, 1-ethylbutyl, n-heptyl, n-octyl, 2-ethylhexyl, isooctyl, tert-octyl, cyclohexyl and cyclooctyl group.

When more than one R exists in said divalent group, all of R may be the same group or different groups.

Examples of the divalent group include cyclohexane-1,4-diyl, 2-cyclohexene-1,4-diyl, 1-cyclohexene-1,4-diyl, 1,4-cyclohexadiene-3,6-diyl, 1,3-cyclohexadiene-1,4-diyl, 1,3-cyclohexanediene-2,5-diyl, 1,4-cyclohexanediene-1,4-diyl, 1,4-phenylene, 2-methylcyclohexane-1,4-diyl, 3-methyl-1,4-phenylene and 3-isopropyl-1,4-phenylene group.

Among the epoxy compounds (1), preferred is an epoxy compound (1) in which Ar¹ and Ar³ independently denote a group represented by the following formula:

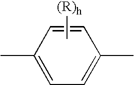

wherein R and h are as defined above, and more preferred is an epoxy compound (1) in which Ar¹ and Ar³ independently denote 1,4-phenylene group, 3-methyl-1,4-phenylene group or 3-isopropyl-1,4-phenylene group.

Preferred is an epoxy compound (1) in which Ar² denotes a group represented by the following formula:

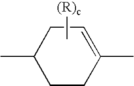

wherein R and c are as defined above, and more preferred is an epoxy compound (1) in which Ar² denotes 1-cyclohexene-1,4-diyl group.

Among these compounds, more preferred is an epoxy compound (1) in which Ar¹ and Ar³ independently denote a group represented by the following formula:

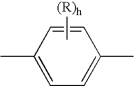

wherein R and h are as defined above, and Ar² denotes a group represented by the following formula:

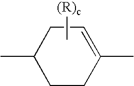

wherein R and c are as defined above, and particularly preferred is an epoxy compound (1) in which Ar¹ and Ar³ independently denote 1,4-phenylene group, 3-methyl-1,4-phenylene group or 3-isopropyl-1,4-phenylene group, and Ar² denotes 1-cyclohexene-1,4-diyl group.

In the above-described formula (1), R¹, R² and R³ are the same or different and each denotes hydrogen atom or an alkyl group having 1 to 8 carbon atoms. Examples of the alkyl group having 1 to 8 carbon atoms include the same groups as those described above.

Q¹ denotes a single bond or a straight-chain alkylene group having 1 to 9 carbon atoms. Examples of the straight-chain alkylene group having 1 to 9 carbon atoms include groups formed by bonding 1 to 9 methylene groups linearly, such as methylene, ethylene, trimethylene, tetramethylene, hexamethylene and nonamethylene group. At least one methylene group composing such a straight-chain alkylene group having 1 to 9 carbon atoms is optionally substituted with an alkyl group having 1 to 8 carbon atoms, and —O— or —N(R⁴)— is optionally inserted between the methylene groups.

Examples of the alkyl group having 1 to 8 carbon atoms include the same groups as those described above.

R⁴ denotes hydrogen atom or an alkyl group having 1 to 8 carbon atoms, and examples of the alkyl group having 1 to 8 carbon atoms include the same groups as those described above.

Examples of the alkylene group in which at least one methylene group is substituted with an alkyl group having 1 to 8 carbon atoms or in which —O— or —N(R⁴)— is inserted between the methylene groups include 2-methyltrimethylene, 1,2-dimethylethylene, 3-oxatetramethylene and 3-oxapentamethylene group.

Q¹ is preferably a single bond.

Q² denotes a straight-chain alkylene group having 1 to 8 carbon atoms and at least one methylene group composing the straight-chain alkylene group is optionally substituted with an alkyl group having 1 to 8 carbon atoms.

Examples of the alkyl group having 1 to 8 carbon atoms and the straight-chain alkylene group having 1 to 8 carbon atoms include the same group as those described above.

Examples of the epoxy compound (1) include
1,4-bis{4-(oxiranylmethoxy)phenyl}cyclohexane,
1-{2-methyl-4-(oxiranylmethoxy)phenyl}-4-{4-(oxiranylmethoxy)phenyl}cyclohexane,
1-{3-methyl-4-(oxiranylmethoxy)phenyl}-4-{4-(oxiranylmethoxy)phenyl}cyclohexane,
1-{3-ethyl-4-(oxiranylmethoxy)phenyl}-4-{4-(oxiranylmethoxy)phenyl}cyclohexane,
1-{3-n-propyl-4-(oxiranylmethoxy)phenyl}-4-{4-(oxiranylmethoxy)phenyl}cyclohexane,
1-{3-isopropyl-4-(oxiranylmethoxy)phenyl}-4-{4-(oxiranylmethoxy)phenyl}cyclohexane,
1-{3-n-butyl-4-(oxiranylmethoxy)phenyl}-4-{4-(oxiranylmethoxy)phenyl}cyclohexane,
1-{3-sec-butyl-4-(oxiranylmethoxy)phenyl}-4-{4-(oxiranylmethoxy)phenyl}cyclohexane,
1-{3-tert-butyl-4-(oxiranylmethoxy)phenyl}-4-{4-(oxiranylmethoxy)phenyl}cyclohexane,
1-{3-n-pentyl-4-(oxiranylmethoxy)phenyl}-4-{4-(oxiranylmethoxy)phenyl}cyclohexane,
1-{3-tert-pentyl-4-(oxiranylmethoxy)phenyl}-4-{4-(oxiranylmethoxy)phenyl}cyclohexane,
1-{3-n-hexyl-4-(oxiranylmethoxy)phenyl}-4-{4-(oxiranylmethoxy)phenyl}cyclohexane,
1-{3-(1-methylpentyl)-4-(oxiranylmethoxy)phenyl}-4-{4-(oxiranylmethoxy)phenyl}cyclohexane,
1-{3-(1-ethylbutyl)-4-(oxiranylmethoxy)phenyl}-4-{4-(oxiranylmethoxy)phenyl}cyclohexane,
1-{3-cyclohexyl-4-(oxiranylmethoxy)phenyl}-4-{4-(oxiranylmethoxy)phenyl}cyclohexane,
1-{3-n-heptyl-(oxiranylmethoxy)phenyl}-4-{4-(oxiranylmethoxy)phenyl}cyclohexane,
1-{3-n-octyl-(oxiranylmethoxy)phenyl}-4-{4-(oxiranylmethoxy)phenyl}cyclohexane,
1-{3-(2-ethylhexyl)-(oxiranylmethoxy)phenyl}-4-{4-(oxiranylmethoxy)phenyl}cyclohexane,
1-{3-tert-octyl-(oxiranylmethoxy)phenyl}-4-{4-(oxiranylmethoxy)phenyl}cyclohexane,
1-{3-cyclooctyl-(oxiranylmethoxy)phenyl}-4-{4-(oxiranylmethoxy)phenyl}cyclohexane,
1-{3-methyl-4-(2-methyl-oxiranylmethoxy)phenyl}-4-{4-(2-methyl-oxiranylmethoxy)phenyl}cyclohexane,
1-{3-methyl-4-(3-methyl-oxiranylmethoxy)phenyl}-4-{4-(3-methyl-oxiranylmethoxy)phenyl}cyclohexane,
1,4-bis{4-(oxiranylmethoxy)phenyl}-1-cyclohexene,
1-{2-methyl-4-(oxiranylmethoxy)phenyl}-4-{4-(oxiranylmethoxy)phenyl}-1-cyclohexene,
1-{3-methyl-4-(oxiranylmethoxy)phenyl}-4-{4-(oxiranylmethoxy)phenyl}-1-cyclohexene,
1-{3-ethyl-4-(oxiranylmethoxy)phenyl}-4-{4-(oxiranylmethoxy)phenyl}-1-cyclohexene,
1-{3-n-propyl-4-(oxiranylmethoxy)phenyl}-4-{4-(oxiranylmethoxy)phenyl}-1-cyclohexene,
1-{3-isopropyl-4-(oxiranylmethoxy)phenyl}-4-{4-(oxiranylmethoxy)phenyl}-1-cyclohexene,
1-{3-n-butyl-4-(oxiranylmethoxy)phenyl}-4-{4-(oxiranylmethoxy)phenyl}-1-cyclohexene,
1-{3-sec-butyl-4-(oxiranylmethoxy)phenyl}-4-{4-(oxiranylmethoxy)phenyl}-1-cyclohexene,
1-{3-tert-butyl-4-(oxiranylmethoxy)phenyl}-4-{4-(oxiranylmethoxy)phenyl}-1-cyclohexene,
1-{3-n-pentyl-4-(oxiranylmethoxy)phenyl}-4-{4-(oxiranylmethoxy)phenyl}-1-cyclohexene,
1-{3-tert-pentyl-4-(oxiranylmethoxy)phenyl}-4-{4-(oxiranylmethoxy)phenyl}-1-cyclohexene,
1-{3-n-hexyl-4-(oxiranylmethoxy)phenyl}-4-{4-(oxiranylmethoxy)phenyl}-1-cyclohexene,
1-{3-(1-methylpentyl)-4-(oxiranylmethoxy)phenyl}-4-{4-(oxiranylmethoxy)phenyl}-1-cyclohexene,
1-{3-(1-ethylbutyl)-4-(oxiranylmethoxy)phenyl}-4-{4-(oxiranylmethoxy)phenyl}-1-cyclohexene,
1-{3-cyclohexyl-4-(oxiranylmethoxy)phenyl}-4-{4-(oxiranylmethoxy)phenyl}-1-cyclohexene,
1-{3-n-heptyl-(oxiranylmethoxy)phenyl}-4-{4-(oxiranylmethoxy)phenyl}-1-cyclohexene,
1-{3-n-octyl-(oxiranylmethoxy)phenyl}-4-{4-(oxiranylmethoxy)phenyl}-1-cyclohexene,
1-{3-(2-ethylhexyl)-(oxiranylmethoxy)phenyl}-4-{4-(oxiranylmethoxy)phenyl}-1-cyclohexene,
1-{3-tert-octyl-(oxiranylmethoxy)phenyl}-4-{4-(oxiranylmethoxy)phenyl}-1-cyclohexene,
1-{3-cyclooctyl-(oxiranylmethoxy)phenyl}-4-{4-(oxiranylmethoxy)phenyl}-1-cyclohexene,
1-{3-methyl-4-(2-methyl-oxiranylmethoxy)phenyl}-4-{4-(2-methyl-oxiranylmethoxy)phenyl}-1-cyclohexene,
1-{3-methyl-4-(3-methyl-oxiranylmethoxy)phenyl}-4-{4-(3-methyl-oxiranylmethoxy)phenyl}-1-cyclohexene,
1,4-bis{4-(oxiranylmethoxy)phenyl}-2-cyclohexene,
1-{2-methyl-4-(oxiranylmethoxy)phenyl}-4-{4-(oxiranylmethoxy)phenyl}-2-cyclohexene,
1-{3-methyl-4-(oxiranylmethoxy)phenyl}-4-{4-(oxiranylmethoxy)phenyl}-2-cyclohexene,
1-{2-methyl-4-(oxiranylmethoxy)phenyl}-4-{4-(oxiranylmethoxy)phenyl}-2-cyclohexene,
1-{3-ethyl-4-(oxiranylmethoxy)phenyl}-4-{4-(oxiranylmethoxy)phenyl}-2-cyclohexene,
1-{3-n-propyl-4-(oxiranylmethoxy)phenyl}-4-{4-(oxiranylmethoxy)phenyl}-2-cyclohexene,
1-{3-isopropyl-4-(oxiranylmethoxy)phenyl}-4-{4-(oxiranylmethoxy)phenyl}-2-cyclohexene,
1-{3-n-butyl-4-(oxiranylmethoxy)phenyl}-4-{4-(oxiranylmethoxy)phenyl}-2-cyclohexene,
1-{3-sec-butyl-4-(oxiranylmethoxy)phenyl}-4-{4-(oxiranylmethoxy)phenyl}-2-cyclohexene,
1-{3-tert-butyl-4-(oxiranylmethoxy)phenyl}-4-{4-(oxiranylmethoxy)phenyl}-2-cyclohexene,
1-{3-n-pentyl-4-(oxiranylmethoxy)phenyl}-4-{4-(oxiranylmethoxy)phenyl}-2-cyclohexene,
1-{3-tert-pentyl-4-(oxiranylmethoxy)phenyl}-4-{4-(oxiranylmethoxy)phenyl}-2-cyclohexene,
1-{3-n-hexyl-4-(oxiranylmethoxy)phenyl}-4-{4-(oxiranylmethoxy)phenyl}-2-cyclohexene,
1-{3-(1-methylpentyl)-4-(oxiranylmethoxy)phenyl}-4-{4-(oxiranylmethoxy)phenyl}-2-cyclohexene,
1-{3-(1-ethylbutyl)-4-(oxiranylmethoxy)phenyl}-4-{4-(oxiranylmethoxy)phenyl}-2-cyclohexene,
1-{3-cyclohexyl-4-(oxiranylmethoxy)phenyl}-4-{4-(oxiranylmethoxy)phenyl}-2-cyclohexene,
1-{3-n-heptyl-(oxiranylmethoxy)phenyl}-4-{4-(oxiranylmethoxy)phenyl}-2-cyclohexene,
1-{3-n-octyl-(oxiranylmethoxy)phenyl}-4-{4-(oxiranylmethoxy)phenyl}-2-cyclohexene,
1-{3-(2-ethylhexyl)-(oxiranylmethoxy)phenyl}-4-{4-(oxiranylmethoxy)phenyl}-2-cyclohexene,
1-{3-tert-octyl-(oxiranylmethoxy)phenyl}-4-{4-(oxiranylmethoxy)phenyl}-2-cyclohexene,
1-{3-cyclooctyl-(oxiranylmethoxy)phenyl}-4-{4-(oxiranylmethoxy)phenyl}-2-cyclohexene,
1-{3-methyl-4-(2-methyl-oxiranylmethoxy)phenyl}-4-{4-(2-methyl-oxiranylmethoxy)phenyl}-2-cyclohexene,
1-{3-methyl-4-(3-methyl-oxiranylmethoxy)phenyl}-4-{4-(3-methyl-oxiranylmethoxy)phenyl}-2-cyclohexene,
1,4-bis{4-(oxiranylmethoxy)phenyl}-2,5-cyclohexadiene,
1-{2-methyl-4-(oxiranylmethoxy)phenyl}-4-{4-(oxiranylmethoxy)phenyl}-2,5-cyclohexadiene,
1-{3-methyl-4-(oxiranylmethoxy)phenyl}-4-{4-(oxiranylmethoxy)phenyl}-2,5-cyclohexadiene,
1-{3-ethyl-4-(oxiranylmethoxy)phenyl}-4-{4-(oxiranylmethoxy)phenyl}-2,5-cyclohexadiene,
1-{3-n-propyl-4-(oxiranylmethoxy)phenyl}-4-{4-(oxiranylmethoxy)phenyl}-2,5-cyclohexadiene,
1-{3-isopropyl-4-(oxiranylmethoxy)phenyl}-4-{4-(oxiranylmethoxy)phenyl}-2,5-cyclohexadiene,
1-{3-n-butyl-4-(oxiranylmethoxy)phenyl}-4-{4-(oxiranylmethoxy)phenyl}-2,5-cyclohexadiene,
1-{3-sec-butyl-4-(oxiranylmethoxy)phenyl}-4-{4-(oxiranylmethoxy)phenyl}-2,5-cyclohexadiene,
1-{3-tert-butyl-4-(oxiranylmethoxy)phenyl}-4-{4-(oxiranylmethoxy)phenyl}-2,5-cyclohexadiene,
1-{3-n-pentyl-4-(oxiranylmethoxy)phenyl}-4-{4-(oxiranylmethoxy)phenyl}-2,5-cyclohexadiene,
1-{3-tert-pentyl-4-(oxiranylmethoxy)phenyl}-4-{4-(oxiranylmethoxy)phenyl}-2,5-cyclohexadiene,
1-{3-n-hexyl-4-(oxiranylmethoxy)phenyl}-4-{4-(oxiranylmethoxy)phenyl}-2,5-cyclohexadiene,
1-{3-(1-methylpentyl)-4-(oxiranylmethoxy)phenyl}-4-{4-(oxiranylmethoxy)phenyl}-2,5-cyclohexadiene,
1-{3-(1-ethylbutyl)-4-(oxiranylmethoxy)phenyl}-4-{4-(oxiranylmethoxy)phenyl}-2,5-cyclohexadiene,
1-{3-cyclohexyl-4-(oxiranylmethoxy)phenyl}-4-{4-(oxiranylmethoxy)phenyl}-2,5-cyclohexadiene,
1-{3-n-heptyl-(oxiranylmethoxy)phenyl}-4-{4-(oxiranylmethoxy)phenyl}-2,5-cyclohexadiene,
1-{3-n-octyl-(oxiranylmethoxy)phenyl}-4-{4-(oxiranylmethoxy)phenyl}-2,5-cyclohexadiene,
1-{3-(2-ethylhexyl)-(oxiranylmethoxy)phenyl}-4-{4-(oxiranylmethoxy)phenyl}-2,5-cyclohexadiene,
1-{3-tert-octyl-(oxiranylmethoxy)phenyl}-4-{4-(oxiranylmethoxy)phenyl}-2,5-cyclohexadiene,
1-{3-cyclooctyl-(oxiranylmethoxy)phenyl}-4-{4-(oxiranylmethoxy)phenyl}-2,5-cyclohexadiene,
1-{3-methyl-4-(2-methyl-oxiranylmethoxy)phenyl}-4-{4-(2-methyl-oxiranylmethoxy)phenyl}-2,5-cyclohexadiene,
1-{3-methyl-4-(3-methyl-oxiranylmethoxy)phenyl}-4-{4-(3-methyl-oxiranylmethoxy)phenyl}-2,5-cyclohexadiene,
1,4-bis{4-(oxiranylmethoxy)phenyl}-1,5-cyclohexadiene,
1-{2-methyl-4-(oxiranylmethoxy)phenyl}-4-{4-(oxiranylmethoxy)phenyl}-1,5-cyclohexadiene,
1-{3-methyl-4-(oxiranylmethoxy)phenyl}-4-{4-(oxiranylmethoxy)phenyl}-1,5-cyclohexadiene,
1-{3-ethyl-4-(oxiranylmethoxy)phenyl}-4-{4-(oxiranylmethoxy)phenyl}-1,5-cyclohexadiene,
1-{3-n-propyl-4-(oxiranylmethoxy)phenyl}-4-{4-(oxiranylmethoxy)phenyl}-1,5-cyclohexadiene,
1-{3-isopropyl-4-(oxiranylmethoxy)phenyl}-4-{4-(oxiranylmethoxy)phenyl}-1,5-cyclohexadiene,
1-{3-n-butyl-4-(oxiranylmethoxy)phenyl}-4-{4-(oxiranylmethoxy)phenyl}-1,5-cyclohexadiene,
1-{3-sec-butyl-4-(oxiranylmethoxy)phenyl}-4-{4-(oxiranylmethoxy)phenyl}-1,5-cyclohexadiene,
1-{3-tert-butyl-4-(oxiranylmethoxy)phenyl}-4-{4-(oxiranylmethoxy)phenyl}-1,5-cyclohexadiene,
1-{3-n-pentyl-4-(oxiranylmethoxy)phenyl}-4-{4-(oxiranylmethoxy)phenyl}-1,5-cyclohexadiene,
1-{3-tert-pentyl-4-(oxiranylmethoxy)phenyl}-4-{4-(oxiranylmethoxy)phenyl}-1,5-cyclohexadiene,
1-{3-n-hexyl-4-(oxiranylmethoxy)phenyl}-4-{4-(oxiranylmethoxy)phenyl}-1,5-cyclohexadiene,
1-{3-(1-methylpentyl)-4-(oxiranylmethoxy)phenyl}-4-{4-(oxiranylmethoxy)phenyl}-1,5-cyclohexadiene,
1-{3-(1-ethylbutyl)-4-(oxiranylmethoxy)phenyl}-4-{4-(oxiranylmethoxy)phenyl}-1,5-cyclohexadiene,
1-{3-cyclohexyl-4-(oxiranylmethoxy)phenyl}-4-{4-(oxiranylmethoxy)phenyl}-1,5-cyclohexadiene,
1-{3-n-heptyl-(oxiranylmethoxy)phenyl}-4-{4-(oxiranylmethoxy)phenyl}-1,5-cyclohexadiene,
1-{3-n-octyl-(oxiranylmethoxy)phenyl}-4-{4-(oxiranylmethoxy)phenyl}-1,5-cyclohexadiene,
1-{3-(2-ethylhexyl)-(oxiranylmethoxy)phenyl}-4-{4-(oxiranylmethoxy)phenyl}-1,5-cyclohexadiene,
1-{3-tert-octyl-(oxiranylmethoxy)phenyl}-4-{4-(oxiranylmethoxy)phenyl}-1,5-cyclohexadiene,
1-{3-cyclooctyl-(oxiranylmethoxy)phenyl}-4-{4-(oxiranylmethoxy)phenyl}-1,5-cyclohexadiene,
1-{3-methyl-4-(2-methyl-oxiranylmethoxy)phenyl}-4-{4-(2-methyl-oxiranylmethoxy)phenyl}-1,5-cyclohexadiene,
1-{3-methyl-4-(3-methyl-oxiranylmethoxy)phenyl}-4-{4-(3-methyl-oxiranylmethoxy)phenyl}-1,5-cyclohexadiene,
1,4-bis{4-(oxiranylmethoxy)phenyl}-1,4-cyclohexadiene,
1-{2-methyl-4-(oxiranylmethoxy)phenyl}-4-{4-(oxiranylmethoxy)phenyl}-1,4-cyclohexadiene,
1-{3-methyl-4-(oxiranylmethoxy)phenyl}-4-{4-(oxiranylmethoxy)phenyl}-1,4-cyclohexadiene,
1-{3-ethyl-4-(oxiranylmethoxy)phenyl}-4-{4-(oxiranylmethoxy)phenyl}-1,4-cyclohexadiene,
1-{3-n-propyl-4-(oxiranylmethoxy)phenyl}-4-{4-(oxiranylmethoxy)phenyl}-1,4-cyclohexadiene,
1-{3-isopropyl-4-(oxiranylmethoxy)phenyl}-4-{4-(oxiranylmethoxy)phenyl}-1,4-cyclohexadiene,
1-{3-n-butyl-4-(oxiranylmethoxy)phenyl}-4-{4-(oxiranylmethoxy)phenyl}-1,4-cyclohexadiene,
1-{3-sec-butyl-4-(oxiranylmethoxy)phenyl}-4-{4-(oxiranylmethoxy)phenyl}-1,4-cyclohexadiene,
1-{3-tert-butyl-4-(oxiranylmethoxy)phenyl}-4-{4-(oxiranylmethoxy)phenyl}-1,4-cyclohexadiene,
1-{3-n-pentyl-4-(oxiranylmethoxy)phenyl}-4-{4-(oxiranylmethoxy)phenyl}-1,4-cyclohexadiene,
1-{3-tert-pentyl-4-(oxiranylmethoxy)phenyl}-4-{4-(oxiranylmethoxy)phenyl}-1,4-cyclohexadiene,
1-{3-n-hexyl-4-(oxiranylmethoxy)phenyl}-4-{4-(oxiranylmethoxy)phenyl}-1,4-cyclohexadiene,
1-{3-(1-methylpentyl)-4-(oxiranylmethoxy)phenyl}-4-{4-(oxiranylmethoxy)phenyl}-1,4-cyclohexadiene,
1-{3-(1-ethylbutyl)-4-(oxiranylmethoxy)phenyl}-4-{4-(oxiranylmethoxy)phenyl}-1,4-cyclohexadiene,
1-{3-cyclohexyl-4-(oxiranylmethoxy)phenyl}-4-{4-(oxiranylmethoxy)phenyl}-1,4-cyclohexadiene,
1-{3-n-heptyl-(oxiranylmethoxy)phenyl}-4-{4-(oxiranylmethoxy)phenyl}-1,4-cyclohexadiene,
1-{3-n-octyl-(oxiranylmethoxy)phenyl}-4-{4-(oxiranylmethoxy)phenyl}-1,4-cyclohexadiene,
1-{3-(2-ethylhexyl)-(oxiranylmethoxy)phenyl}-4-{4-(oxiranylmethoxy)phenyl}-1,4-cyclohexadiene,
1-{3-tert-octyl-(oxiranylmethoxy)phenyl}-4-{4-(oxiranylmethoxy)phenyl}-1,4-cyclohexadiene,
1-{3-cyclooctyl-(oxiranylmethoxy)phenyl}-4-{4-(oxiranylmethoxy)phenyl}-1,4-cyclohexadiene,
1-{3-methyl-4-(2-methyl-oxiranylmethoxy)phenyl}-4-{4-(2-methyl-oxiranylmethoxy)phenyl}-1,4-cyclohexadiene,
1-{3-methyl-4-(3-methyl-oxiranylmethoxy)phenyl}-4-{4-(3-methyl-oxiranylmethoxy)phenyl}-1,4-cyclohexadiene,
1,4-bis{4-(oxiranylmethoxy)phenyl}-1,3-cyclohexadiene,
1-{2-methyl-4-(oxiranylmethoxy)phenyl}-4-{4-(oxiranylmethoxy)phenyl}-1,3-cyclohexadiene,
1-{3-methyl-4-(oxiranylmethoxy)phenyl}-4-{4-(oxiranylmethoxy)phenyl}-1,3-cyclohexadiene,
1-{3-ethyl-4-(oxiranylmethoxy)phenyl}-4-{4-(oxiranylmethoxy)phenyl}-1,3-cyclohexadiene,
1-{3-n-propyl-4-(oxiranylmethoxy)phenyl}-4-{4-(oxiranylmethoxy)phenyl}-1,3-cyclohexadiene,
1-{3-isopropyl-4-(oxiranylmethoxy)phenyl}-4-{4-(oxiranylmethoxy)phenyl}-1,3-cyclohexadiene,
1-{3-n-butyl-4-(oxiranylmethoxy)phenyl}-4-{4-(oxiranylmethoxy)phenyl}-1,3-cyclohexadiene,
1-{3-sec-butyl-4-(oxiranylmethoxy)phenyl}-4-{4-(oxiranylmethoxy)phenyl}-1,3-cyclohexadiene,
1-{3-tert-butyl-4-(oxiranylmethoxy)phenyl}-4-{4-(oxiranylmethoxy)phenyl}-1,3-cyclohexadiene,
1-{3-n-pentyl-4-(oxiranylmethoxy)phenyl}-4-{4-(oxiranylmethoxy)phenyl}-1,3-cyclohexadiene,
1-{3-tert-pentyl-4-(oxiranylmethoxy)phenyl}-4-{4-(oxiranylmethoxy)phenyl}-1,3-cyclohexadiene,
1-{3-n-hexyl-4-(oxiranylmethoxy)phenyl}-4-{4-(oxiranylmethoxy)phenyl}-1,3-cyclohexadiene,
1-{3-(1-methylpentyl)-4-(oxiranylmethoxy)phenyl}-4-{4-(oxiranylmethoxy)phenyl}-1,3-cyclohexadiene,
1-{3-(1-ethylbutyl)-4-(oxiranylmethoxy)phenyl}-4-{4-(oxiranylmethoxy)phenyl}-1,3-cyclohexadiene,
1-{3-cyclohexyl-4-(oxiranylmethoxy)phenyl}-4-{4-(oxiranylmethoxy)phenyl}-1,3-cyclohexadiene,
1-{3-n-heptyl-(oxiranylmethoxy)phenyl}-4-{4-(oxiranylmethoxy)phenyl}-1,3-cyclohexadiene,
1-{3-n-octyl-(oxiranylmethoxy)phenyl}-4-{4-(oxiranylmethoxy)phenyl}-1,3-cyclohexadiene,
1-{3-(2-ethylhexyl)-(oxiranylmethoxy)phenyl}-4-{4-(oxiranylmethoxy)phenyl}-1,3-cyclohexadiene,
1-{3-tert-octyl-(oxiranylmethoxy)phenyl}-4-{4-(oxiranylmethoxy)phenyl}-1,3-cyclohexadiene,
1-{3-cyclooctyl-(oxiranylmethoxy)phenyl}-4-{4-(oxiranylmethoxy)phenyl}-1,3-cyclohexadiene,
1-{3-methyl-4-(2-methyl-oxiranylmethoxy)phenyl}-4-{4-(2-methyl-oxiranylmethoxy)phenyl}-1,3-cyclohexadiene,
1-{3-methyl-4-(3-methyl-oxiranylmethoxy)phenyl}-4-{4-(3-methyl-oxiranylmethoxy)phenyl}-1,3-cyclohexadiene,
1,4-bis{4-(oxiranylmethoxy)phenyl}benzene,
1-{2-methyl-4-(oxiranylmethoxy)phenyl}-4-{4-(oxiranylmethoxy)phenyl}benzene,
1-{3-methyl-4-(oxiranylmethoxy)phenyl}-4-{4-(oxiranylmethoxy)phenyl}benzene,
1-{3-ethyl-4-(oxiranylmethoxy)phenyl}-4-{4-(oxiranylmethoxy)phenyl}benzene,
1-{3-n-propyl-4-(oxiranylmethoxy)phenyl}-4-{4-(oxiranylmethoxy)phenyl}benzene,
1-{3-isopropyl-4-(oxiranylmethoxy)phenyl}-4-{4-(oxiranylmethoxy)phenyl}benzene,
1-{3-n-butyl-4-(oxiranylmethoxy)phenyl}-4-{4-(oxiranylmethoxy)phenyl}benzene,
1-{3-sec-butyl-4-(oxiranylmethoxy)phenyl}-4-{4-(oxiranylmethoxy)phenyl}benzene,
1-{3-tert-butyl-4-(oxiranylmethoxy)phenyl}-4-{4-(oxiranylmethoxy)phenyl}benzene,
1-{3-n-pentyl-4-(oxiranylmethoxy)phenyl}-4-{4-(oxiranylmethoxy)phenyl}benzene,
1-{3-tert-pentyl-4-(oxiranylmethoxy)phenyl}-4-{4-(oxiranylmethoxy)phenyl}benzene,
1-{3-n-hexyl-4-(oxiranylmethoxy)phenyl}-4-{4-(oxiranylmethoxy)phenyl}benzene,
1-{3-(1-methylpentyl)-4-(oxiranylmethoxy)phenyl}-4-{4-(oxiranylmethoxy)phenyl}benzene,
1-{3-(1-ethylbutyl)-4-(oxiranylmethoxy)phenyl}-4-{4-(oxiranylmethoxy)phenyl}benzene,
1-{3-cyclohexyl-4-(oxiranylmethoxy)phenyl}-4-{4-(oxiranylmethoxy)phenyl}benzene,
1-{3-n-heptyl-(oxiranylmethoxy)phenyl}-4-{4-(oxiranylmethoxy)phenyl}benzene,
1-{3-n-octyl-(oxiranylmethoxy)phenyl}-4-{4-(oxiranylmethoxy)phenyl}benzene,
1-{3-(2-ethylhexyl)-(oxiranylmethoxy)phenyl}-4-{4-(oxiranylmethoxy)phenyl}benzene,
1-{3-tert-octyl-(oxiranylmethoxy)phenyl}-4-{4-(oxiranylmethoxy)phenyl}benzene,
1-{3-cyclooctyl-(oxiranylmethoxy)phenyl}-4-{4-(oxiranylmethoxy)phenyl}benzene,
1-{3-methyl-4-(2-methyl-oxiranylmethoxy)phenyl}-4-{4-(2-methyl-oxiranylmethoxy)phenyl}benzene,
1-{3-methyl-4-(3-methyl-oxiranylmethoxy)phenyl}-4-{4-(3-methyl-oxiranylmethoxy)phenyl}benzene,
1,4-bis{4-(oxiranylmethoxyethoxy)phenyl}cyclohexane,
1-{3-methyl-4-(oxiranylmethoxyethoxy)phenyl}-4-{4-(oxiranylmethoxyethoxy)phenyl}cyclohexane,
1,4-bis{4-(3-oxiranylpropoxy)phenyl}cyclohexane,
1-{3-methyl-4-(3-oxiranylpropoxy)phenyl}-4-{4-(3-oxiranylpropoxy)phenyl}cyclohexane,
1,4-bis{4-(4-oxiranylbutoxy)phenyl}cyclohexane,
1-{3-methyl-4-(4-oxiranylbutoxy)phenyl}-4-{4-(4-oxiranylbutoxy)phenyl}cyclohexane,
1,4-bis{4-(5-oxiranylpentyloxy)phenyl}cyclohexane,
1-{3-methyl-4-(5-oxiranylpentyloxy)phenyl}-4-{4-(5-oxiranylpentyloxy)phenyl}cyclohexane,
1,4-bis{4-(6-oxiranylhexyloxy)phenyl}cyclohexane,
1-{3-methyl-4-(6-oxiranylhexyloxy)phenyl}-4-{4-(6-oxiranylhexyloxy)phenyl}cyclohexane,
1,4-bis{4-(8-oxiranyloctyloxy)phenyl}cyclohexane,
1-{3-methyl-4-(8-oxiranyloctyloxy)phenyl}-4-{4-(8-oxiranyloctyloxy)phenyl}cyclohexane,
1,4-bis{4-(oxiranylmethoxyethoxy)phenyl}cyclohexane,
1-{3-methyl-4-(oxiranylmethoxyethoxy)phenyl}-4-{4-(oxiranylmethoxyethoxy)phenyl}cyclohexane,
1,4-bis{4-(2-methyl-oxiranyl)methoxyethoxyphenyl}cyclohexane,
1-{3-methyl-4-(2-methyl-oxiranyl)methoxyethoxyphenyl}-4-{4-(2-methyl-oxiranyl)methoxyethoxyphenyl}cyclohexane,
1,4-bis{4-(3-methyl-oxiranyl)methoxyethoxyphenyl}cyclohexane,
1-{3-methyl-4-(3-methyl-oxiranyl)methoxyethoxyphenyl}-4-{4-(3-methyl-oxiranyl)methoxyethoxyphenyl}cyclohexane,
1,4-bis{4-(oxiranylethoxy)phenyl}-1-cyclohexene,
1-{3-methyl-4-(oxiranylethoxy)phenyl}-4-{4-(oxiranylethoxy)phenyl}-1-cyclohexene,
1,4-bis{4-(3-oxiranylpropoxy)phenyl}-1-cyclohexene,
1-{3-methyl-4-(3-oxiranylpropoxy)phenyl}-4-{4-(3-oxiranylpropoxy)phenyl}-1-cyclohexene,
1,4-bis{4-(4-oxiranylbutoxy)phenyl}-1-cyclohexene,
1-{3-methyl-4-(4-oxiranylbutoxy)phenyl}-4-{4-(4-oxiranylbutoxy)phenyl}-1-cyclohexene,
1,4-bis{4-(5-oxiranylpentyloxy)phenyl}-1-cyclohexene,
1-{3-methyl-4-(5-oxiranylpentyloxy)phenyl}-4-{4-(5-oxiranylpentyloxy)phenyl}-1-cyclohexene,
1,4-bis{4-(6-oxiranylhexyloxy)phenyl}-1-cyclohexene,
1-{3-methyl-4-(6-oxiranylhexyloxy)phenyl}-4-{4-(6-oxiranylhexyloxy)phenyl}-1-cyclohexene,
1,4-bis{4-(8-oxiranyloctyloxy)phenyl}-1-cyclohexene,
1-{3-methyl-4-(8-oxiranyloctyloxy)phenyl}-4-{4-(8-oxiranyloctyloxy)phenyl}-1-cyclohexene,
1,4-bis{4-(oxiranylmethoxyethoxy)phenyl}-1-cyclohexene,
1-{3-methyl-4-(oxiranylmethoxyethoxy)phenyl}-4-{4-(oxiranylmethoxyethoxy)phenyl}-1-cyclohexene,
1,4-bis{4-(2-methyl-oxiranyl)methoxyethoxyphenyl}-1-cyclohexene,
1-{3-methyl-4-(2-methyl-oxiranyl)methoxyethoxyphenyl}-4-{4-(2-methyl-oxiranyl)methoxyethoxyphenyl}-1-cyclohexene,
1,4-bis{4-(3-methyl-oxiranyl)methoxyethoxyphenyl}-1-cyclohexene,
1-{3-methyl-4-(3-methyl-oxiranyl)methoxyethoxyphenyl}-4-{4-(3-methyl-oxiranyl)methoxyethoxyphenyl}-1-cyclohexene,
1,4-bis{4-(oxiranylmethoxyethoxy)phenyl}benzene,
1-{3-methyl-4-(oxiranylmethoxyethoxy)phenyl}-4-{4-(oxiranylmethoxyethoxy)phenyl}benzene,
1,4-bis{4-(3-oxiranylpropoxy)phenyl}benzene,
1-{3-methyl-4-(3-oxiranylpropoxy)phenyl}-4-{4-(3-oxiranylpropoxy)phenyl}benzene,
1,4-bis{4-(4-oxiranylbutoxy)phenyl}benzene,
1-{3-methyl-4-(4-oxiranylbutoxy)phenyl}-4-{4-(4-oxiranylbutoxy)phenyl}benzene,
1,4-bis{4-(5-oxiranylpentyloxy)phenyl}benzene,
1-{3-methyl-4-(5-oxiranylpentyloxy)phenyl}-4-{4-(5-oxiranylpentyloxy)phenyl}benzene,
1,4-bis{4-(6-oxiranylhexyloxy)phenyl}benzene,
1-{3-methyl-4-(6-oxiranylhexyloxy)phenyl}-4-{4-(6-oxiranylhexyloxy)phenyl}benzene,
1,4-bis{4-(8-oxiranyloctyloxy)phenyl}benzene,
1-{3-methyl-4-(8-oxiranyloctyloxy)phenyl}-4-{4-(8-oxiranyloctyloxy)phenyl}benzene,
1,4-bis{4-(oxiranylmethoxyethoxy)phenyl}benzene,
1-{3-methyl-4-(oxiranylmethoxyethoxy)phenyl}-4-{4-(oxiranylmethoxyethoxy)phenyl}benzene,
1,4-bis{4-(2-methyl-oxiranyl)methoxyethoxyphenyl}benzene,
1-{3-methyl-4-(2-methyl-oxiranyl)methoxyethoxyphenyl}-4-{4-(2-methyl-oxiranyl)methoxyethoxyphenyl}benzene,
1,4-bis{4-(3-methyl-oxiranyl)methoxyethoxyphenyl}benzene and
1-{3-methyl-4-(3-methyl-oxiranyl)methoxyethoxyphenyl}-4-{4-(3-methyl-oxiranyl)methoxyethoxyphenyl}benzene.

As described in EP 1698625 A1, a crude product of an epoxy compound, which contains the epoxy compound (1) as a main component, can be produced, for example, by a method comprising reacting a dihydroxy compound represented by the formula (2):

HO-Q¹-Ar¹—Ar²—Ar³-Q¹-OH (2)

wherein Q¹, Ar¹, Ar² and Ar³ are as defined above (hereinafter abbreviated to the dihydroxy compound (2)) with a compound represented by the formula (3):

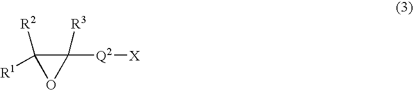

wherein R¹, R², R³ and Q² are as defined above, and X denotes a halogen atom (hereinafter abbreviated to the compound (3)) in the presence of a base such as sodium hydroxide, concentrating the obtained reaction mixture to remove the unreacted compound (3), adding a solvent, and optionally washing with water. A crude product of an epoxy compound containing the epoxy compound (1) as a main component can also be produced by a method comprising reacting the dihydroxy compound (2) with the compound (3) in the presence of at least one selected from the group consisting of an amine compound and an ammonium salt, mixing the obtained reaction mixture with an inorganic base to conduct reaction, concentrating the obtained reaction mixture to remove the unreacted compound (3), adding a solvent, and optionally washing with water. When insolubles such as an inorganic salt are precipitated in the obtained crude product, the insolubles are preferably removed by filtration.

When a crude product of an epoxy compound containing the epoxy compound (1) as a main component is produced by reacting the dihydroxy compound (2) with the compound (3) in the presence of at least one selected from the group consisting of an amine compound and an ammonium salt, and mixing the obtained reaction mixture with an inorganic base to further conduct reaction, the inorganic base is usually added to the reaction mixture after dihydroxy compound (2) disappeared as a result of the reaction of the dihydroxy compound (2) and the compound (3). It is preferred to add the inorganic base to the reaction mixture after the dihydroxy compound (2) disappeared and after 50% or more of the compound obtained by reacting one molecule of the obtained dihydroxy compound (2) and one molecule of the compound (3) disappeared. The content of the compound obtained by reacting one molecule of the obtained dihydroxy compound (2) and one molecule of the compound (3) in the reaction mixture can be checked by a conventional analytical means such as liquid chromatography.

Examples of the amine compound include tertiary amines such as trimethylamine, triethylamine, tri-n-propylamine, tri-n-butylamine, N,N-dimethylaniline, N-methylpyrrolidine, N-methylpiperidine and N-methylmorpholine. Examples of the ammonium salt include quaternary ammonium halides such as tetraethylammonium chloride, tetra-n-butylammonium chloride, benzyltriethylammonium chloride, tetraethylammonium bromide and tetra-n-butylammonium bromide. The amount thereof is usually from 0.005 to 20 mol based on 1 mol of the dihydroxy compound (2).

The reaction of the dihydroxy compound (2) and the compound (3) may be carried out in the absence or presence of a solvent. Examples of the solvent include aliphatic hydrocarbon solvents such as n-pentane, n-hexane and n-heptanecyclohexane; aromatic hydrocarbon solvents such as toluene and xylene; alcohol solvents such as methanol, ethanol, n-propanol and isopropanol; glycol solvents such as ethylene glycol; ketone solvents such as acetone and methyl ethyl ketone; nitrile solvents such as acetonitrile; ether solvents such as diethyl ether, tert-butyl methyl ether, 1,4-dioxane and tetrahydrofuran; and amide solvents such as N,N-dimethylformamide. The compound (3) may be used as the solvent.

The reaction temperature is usually from 10 to 100° C.

Examples of the inorganic base include alkali metal hydroxides such as lithium hydroxide, sodium hydroxide and potassium hydroxide; and alkali metal carbonates such as sodium carbonate and potassium carbonate.

The amount of the inorganic base is usually from 0.2 to 20 mol based on 1 mol of the dihydroxy compound (2) used in the reaction of the dihydroxy compound (2) and the compound (3).

The temperature at which the inorganic base is mixed with the reaction mixture, is usually from 0 to 100° C.

As described above, a crude product of an epoxy compound containing the epoxy compound (1) obtained by reacting the dihydroxy compound (2) and the compound (3) as a main component is usually used as the crude product of the epoxy compound containing the epoxy compound (1) as a main component in the present invention. A crude product containing 70% by weight or more of epoxy compound (1) is preferred and a crude product containing 85% by weight or more of epoxy compound (1) is more preferred.

The crude product usually contains the dihydroxy compound (2) as a raw material used, or reaction intermediates. When a dihydroxy compound represented by the formula (4):

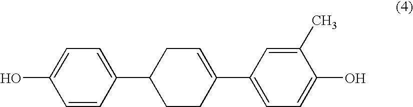

is used as the dihydroxy compound (2) and epichlorohydrin is used as the compound (3), a crude product containing an epoxy compound represented by the formula (5):

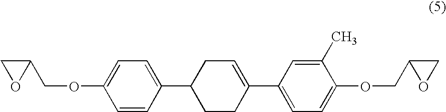

as a main component is obtained. The crude product contains a dihydroxy compound represented by the formula (4) as a raw material, a compound represented by the following formula (6)

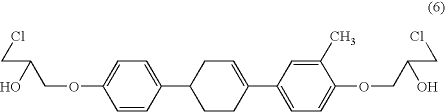

and a compound represented by the formula (7).

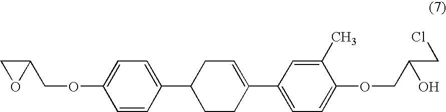

Examples of the solvent of the solution containing a crude product of the epoxy compound include aliphatic ketone solvents such as acetone, methyl ethyl ketone, diethyl ketone and methyl isobutyl ketone; ether solvents such as diethyl ether, tert-butyl methyl ether, 1,4-dioxane and tetrahydrofuran; aromatic hydrocarbon solvents such as benzene, toluene, ethylbenzene, xylene, cumene, cymene and chlorobenzene; and nitrile solvents such as acetonitrile and propionitrile; or a mixed solvent thereof. At least one selected from the group consisting of aliphatic ketone solvents, ether solvents and aromatic hydrocarbon solvents is preferred. The solution may contain aliphatic hydrocarbon solvents such as n-pentane, n-hexane, 1-hexane, n-heptane, 1-octane, n-octane, n-decane, cyclopentane and cyclohexane.

When the amount of the solvent is too large, the yield of the epoxy compound (1) decreases. In contrast, when the amount is too small, adhesion thereof to the inside of a reaction vessel by scaling is likely to occur. Therefore, a saturation temperature T° C. of the epoxy compound (1) in the solution is adjusted within a range from 30 to 60° C. according to the kind and amount of the epoxy compound (1) and the kind of the solvent. The concentration of the epoxy compound (1) in the solution is preferably from 10 to 40% by weight. As used herein, the saturation temperature TOC means a temperature when the solution is cooled to precipitate crystals of the epoxy compound (1) and then the entire precipitated crystals are dissolved by heating the solution in which crystals of the obtained epoxy compound (1) are precipitated.

The step (A) is a step of cooling a solution containing a crude product of the epoxy compound, which contains the epoxy compound as a main component, to an internal temperature T¹° C. to precipitate crystals of the epoxy compound (1), thus obtaining a suspension.

The cooling temperature T¹° C. of the solution may be a temperature which is the saturation temperature T° C. or lower and at which crystals of the epoxy compound (1) can be precipitated. While it varies depending on the content of the epoxy compound (1) and the kind of the organic solvent, when the concentration of the epoxy compound (1) in the solution is from 10 to 40% by weight, the cooling temperature T¹° C. is preferably a temperature which satisfies the following formula (III):

T¹<T−5 (III)

and specifically, it is from 10 to 30° C., and preferably from 20 to 30° C.

If necessary, seed crystals may be added in the solution. Preferably, seed crystals are added after the temperature of the solution became saturation temperature T° C. or lower. More preferably, seed crystals are added at the stage where the temperature of the solution is within a range from T° C. to (T−20) ° C. and crystals of the epoxy compound (1) are not precipitated.

It is preferred that the suspension cooled to the temperature T¹° C. is maintained at the same temperature for about 1 to 3 hours while stirring.

Thus, there can be obtained a suspension in which usually 10 to 80% by weight, and preferably 20 to 60% by weight of the epoxy compound (1) contained in the solution is precipitated in the form of crystals.

Subsequently, the step (B) of heating the suspension obtained in the step (A) to a temperature T²° C., which satisfies the formula (1):

T¹<T²<T (I)

wherein T denotes a saturation temperature of an epoxy compound in the solution containing a crude product of the epoxy compound used in the step (A), will be described.

The temperature T² may be a temperature which is lower than a saturation temperature T° C. of the epoxy compound in the solution containing a crude product of the epoxy compound used in the step (A), which is higher than cooling temperature T¹ in the step (A), and at which a portion of crystals of the epoxy compound (1) in the suspension can be dissolved. Preferably, the temperature is a temperature at which the suspension wherein about 10 to 50% by weight of the epoxy compound (1) in the used solution is precipitated in the form of crystals is obtained. Specifically, when the concentration of the epoxy compound (1) is from 10 to 40% by weight, the temperature T² preferably satisfies the following formula (IV).

T−5<T²<T (IV)

Practically, the temperature T² is preferably from 30 to 60° C., and more preferably from 35 to 45° C.

After heating to T²° C., the suspension is preferably maintained at the same temperature for about 1 to 4 hours while stirring.

Finally, the step (C) of cooling the suspension heated to T²° C. in the step (B) to a temperature T³° C., which satisfies the following formula (II), will be described.

T³<T¹ (II)

While the temperature T³ may be lower than the cooling temperature T¹ in the step (A), it is preferably a temperature at which 75% by weight or more of the epoxy compound (1) in the used solution is precipitated in the form of crystals. Specifically, when the concentration of the epoxy compound (1) is from 10 to 40% by weight, the temperature T³ preferably satieties the following formula (V).

−10<T³<T−5 (V)

From the point of the view of viscosity of the obtained suspension, the temperature T³ is preferably from −10 to 20° C., and more preferably from 0 to 10° C.

Crystals of the epoxy compound (1) can be isolated by filtering the suspension thus obtained. It is preferred that the suspension is cooled to T³° C. and maintained at the same temperature for about 1 to 10 hours while stirring, and then filtered.

Purity of the obtained epoxy compound (1) can be analyzed by a conventional analytical means such as high-performance liquid chromatography.

### EXAMPLE

Hereinafter, the present invention is further illustrated in detail by referring to Examples, but the present invention is not limited to Examples. Analysis was carried out by high-performance liquid chromatography and purity of the obtained epoxy compound was calculated by a high-performance liquid chromatography area percentage method.

### Reference Example 1

25.0 g of 1-(3-methyl-4-hydroxyphenyl)-4-(4-hydroxyphenyl)-1-cyclohexene, 244.9 g of epichlorohydrin and 5.8 g of tetra-n-butylammonium bromide were placed in a reaction vessel equipped with a thermometer, a stirrer and a condenser tube. Under a nitrogen atmosphere, the obtained mixture was reacted by heating at 50° C. for 10 hours while stirring. The reaction mixture was analyzed by high-performance liquid chromatography. As a result, 1-(3-methyl-4-hydroxyphenyl)-4-(4-hydroxyphenyl)-1-cyclohexene disappeared and an area percentage value of a compound obtained by reacting one molecule of 1-(3-methyl-4-hydroxyphenyl)-4-(4-hydroxyphenyl)-1-cyclohexene and one molecule of epichlorohydrinone was 0.41%. The obtained reaction mixture was cooled to room temperature and 11.1 g of sodium hydroxide was added thereto, followed by further reaction at room temperature for 2 hours to obtain the reaction mixture containing 1-(3-methyl-4-oxiranylmethoxyphenyl)-4-(4-oxiranylmethoxyphenyl)-1-cyclohexene. Under reduced pressure conditions, the reaction mixture was concentrated at 50° C. to remove the unreacted epichlorohydrin. To 78.4 g of the obtained concentrated residue, 245.7 g of methyl isobutyl ketone and 100.8 g of ion-exchange water were added, followed by stirring at 50° C. for 30 minutes. After standing, the organic layer and the aqueous layer were separated. The obtained organic layer was washed with 81.1 g of ion-exchange water and concentrated under reduced pressure conditions at 50° C., and then precipitated insolubles were removed by filtration to obtain 250.1 g of a methyl isobutyl ketone solution of 1-(3-methyl-4-oxiranylmethoxyphenyl)-4-(4-oxiranylmethoxyphenyl)-1-cyclohexene (area percentage value of 1-(3-methyl-4-oxiranylmethoxyphenyl)-4-(4-oxiranylmethoxyphenyl)-1-cyclohexene: 96.2%). Saturation temperature was 42° C.

### Example 1

250.1 g of a methyl isobutyl ketone solution containing 1-(3-methyl-4-oxiranylmethoxyphenyl)-4-(4-oxiranylmethoxyphenyl)-1-cyclohexene obtained in Reference Example 1 was cooled to 20° C. to precipitate crystals, and thus a suspension was obtained. The obtained suspension was heated to 40° C. and then maintained at the same temperature for 3 hours. The suspension was cooled to 10° C. over 7 hours and then maintained at the same temperature for 2 hours. The obtained suspension was filtered. The filtration time was within one minute and filterability of crystals was extremely excellent. The obtained crystal was washed three times with 20 g of methyl isobutyl ketone adjusted to 10° C. and then dried under reduced pressure conditions at 50° C. to obtain 27.3 g of 1-(3-methyl-4-oxiranylmethoxyphenyl)-4-(4-oxiranylmethoxyphenyl)-1-cyclohexene as white crystals. Area percentage value: 98.8%, yield: 77.8%.

### Example 2

218.8 g of a methyl isobutyl ketone solution (saturation temperature: 42° C.) containing 1-(3-methyl-4-oxiranylmethoxyphenyl)-4-(4-oxiranylmethoxyphenyl)-1-cyclohexene obtained in the same manner as in Reference Example 1 was cooled to 20° C. to precipitate crystals and thus a suspension was obtained. The obtained suspension was heated to 38° C. and then maintained at the same temperature for 3 hours. The suspension was cooled to 10° C. over 3.5 hours and then maintained at the same temperature for 2 hours. The obtained suspension was filtered. The filtration time was within one minute and filterability of crystals was extremely excellent. The obtained crystal was washed three times with 20 g of methyl isobutyl ketone adjusted to 10° C. and then dried under reduced pressure conditions at 50° C. to obtain 24.6 g of 1-(3-methyl-4-oxiranylmethoxyphenyl)-4-(4-oxiranylmethoxyphenyl)-1-cyclohexene as white crystals. Area percentage value: 98.7%, yield: 70.4%.

### Example 3

217.7 g of a methyl isobutyl ketone solution (saturation temperature: 42° C.) containing 1-(3-methyl-4-oxiranylmethoxyphenyl)-4-(4-oxiranylmethoxyphenyl)-1-cyclohexene obtained in the same manner as in Reference Example 1 and then cooled to 20° C. to precipitate crystals, and thus a suspension was obtained. The obtained suspension was heated to 35° C. and then maintained at the same temperature for 3 hours. The suspension was cooled to 10° C. over 7 hours and then maintained at the same temperature for 2 hours. The obtained suspension was filtered. The filtration time was within one minute and filterability of crystals was extremely excellent. The obtained crystal was washed three times with 20 g of methyl isobutyl ketone adjusted to 10° C. and then dried under reduced pressure conditions at 50° C. to obtain 23.8 g of 1-(3-methyl-4-oxiranylmethoxyphenyl)-4-(4-oxiranylmethoxyphenyl)-1-cyclohexene as white crystals. Area percentage value: 97.8%, yield: 68.0%.

### Reference Example 2

In the same manner as in Reference Example 1, the reaction of 1-(3-methyl-4-hydroxyphenyl)-4-(4-hydroxyphenyl)-1-cyclohexene and epichlorohydrin was carried out to obtain 94.1 g of a concentrated residue. To the obtained concentrated residue, 200 g of toluene and 100.0 g of ion-exchange water were added, followed by stirring at 50° C. for 30 minutes. After standing, the organic layer and the aqueous layer were separated. The obtained organic layer was washed twice with ion-exchange water and then concentrated under reduced pressure conditions at 50° C. The precipitated insolubles were removed by filtration to obtain 218.8 g of a toluene solution containing 1-(3-methyl-4-oxiranylmethoxyphenyl)-4-(4-oxiranylmethoxyphenyl)-1-cyclohexene. Saturation temperature was 40° C.

### Example 4

218.8 g of a toluene solution containing 1-(3-methyl-4-oxiranylmethoxyphenyl)-4-(4-oxiranylmethoxyphenyl)-1-cyclohexene obtained in Reference Example 2 was cooled to 20° C. to precipitate crystals, thus obtaining a suspension. The obtained suspension was heated to 38° C. and then maintained at the same temperature for 3 hours. The suspension was cooled to 10° C. over 3.5 hours and then maintained at the same temperature for 2 hours. The obtained suspension was filtered. The filtration time was within one minute and filterability of crystals was extremely excellent. The obtained crystal was washed three times with 20 g of toluene adjusted to 10° C. and then dried under reduced pressure conditions at 50° C. to obtain 17.9 g of 1-(3-methyl-4-oxiranylmethoxyphenyl)-4-(4-oxiranylmethoxyphenyl)-1-cyclohexene as white crystals. Area percentage value: 98.1%, yield: 51.4%.

### Comparative Example 1

260.0 g of a methyl isobutyl ketone solution containing 1-(3-methyl-4-oxiranylmethoxyphenyl)-4-(4-oxiranylmethoxyphenyl)-1-cyclohexene obtained in the same manner as in Reference Example 1 was cooled to 10° C. over 3.5 hours and then maintained at the same temperature for 2 hours. The obtained suspension was filtered. As a result, the filtration time was 30 minutes or more and filterability was very poor. The obtained crystals were washed with 20 g of methyl isobutyl ketone adjusted to 10° C. and then dried under reduced pressure conditions at 50° C. to obtain 27.3 g of 1-(3-methyl-4-oxiranylmethoxyphenyl)-4-(4-oxiranylmethoxyphenyl)-1-cyclohexene as white crystals. Area percentage value: 97.5%, yield: 74.8%.

### Comparative Example 2

260.8 g of a methyl isobutyl ketone solution containing 1-(3-methyl-4-oxiranylmethoxyphenyl)-4-(4-oxiranylmethoxyphenyl)-1-cyclohexene obtained in the same manner as in Reference Example 1 was cooled to 10° C. over 7 hours and then maintained at the same temperature for 2 hours. The obtained suspension was filtered. As a result, the filtration time was 30 minutes and filterability was very poor. The obtained crystals were washed with 20 g of methyl isobutyl ketone adjusted to 10° C. was dried under reduced pressure conditions at 50° C. to obtain 27.3 g of 1-(3-methyl-4-oxiranylmethoxyphenyl)-4-(4-oxiranylmethoxyphenyl)-1-cyclohexene as white crystals. Area percentage value: 97.4%, yield: 74.2%.

### INDUSTRIAL APPLICABILITY

According to the present invention, an epoxy compound represented by the formula (3), which can form a cured resin that is useful as an insulating material requiring a high thermal conductivity, can be obtained with higher purity and it is industrially advantageous.

1. A process for crystallizing an epoxy compound, which comprises the steps of:
(A) cooling a solution containing a crude product of an epoxy compound, which contains as a main component the epoxy compound represented by the formula (1): 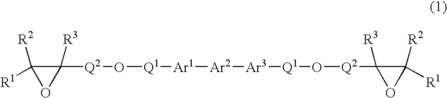
wherein
Ar¹, Ar² and Ar³ are the same or different and each denotes any one of divalent groups represented by the following formulas: 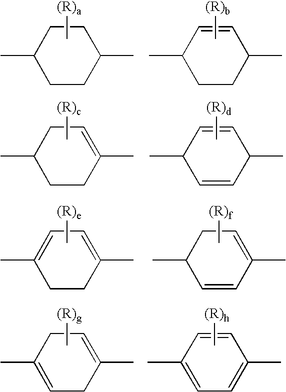
in which R denotes an alkyl group having 1 to 8 carbon atoms, a denotes an integer of 0 to 8, b, e and g denote an integer of 0 to 6, c denotes an integer of 0 to 7, d and h denote an integer of 0 to 4, and f denotes an integer of 0 to 5, and when more than one R exists in said divalent group, all of R may be the same group or different groups;
R¹, R² and R³ are the same or different and each denotes hydrogen atom or an alkyl group having 1 to 8 carbon atoms;
Q¹ denotes a single bond or a straight-chain alkylene group having 1 to 9 carbon atoms, in which at least one methylene group composing the straight-chain alkylene group is optionally substituted with an alkyl group having 1 to 8 carbon atoms and —O— or —N(R⁴)— is optionally inserted between the methylene groups, in which R⁴ denotes hydrogen atom or an alkyl group having 1 to 8 carbon atoms; and
Q² denotes a straight-chain alkylene group having 1 to 8 carbon atoms, in which at least one methylene group composing the straight-chain alkylene group is optionally substituted with an alkyl group having 1 to 8 carbon atoms;
to an internal temperature T¹° C. to precipitate crystals of the epoxy compound represented by the formula (1), thus obtaining a suspension;
(B) heating the suspension obtained in the above-described step (A) to a temperature T²° C. which satisfies the formula (I):
T¹<T²<T (I)
wherein T denotes a saturation temperature of the epoxy compound in the solution containing a crude product of the epoxy compound used in the above-described step (A); and
(C) cooling the suspension heated to the temperature T²° C. in the above-described step (B) to a temperature T³° C. which satisfies the following formula (II).
T³<T¹ (II)

2. The process for crystallizing an epoxy compound according to claim 1, wherein a solvent of the solution containing a crude product of the epoxy compound is at least one selected from the group consisting of an aliphatic ketone solvent, an ether solvent and an aromatic hydrocarbon solvent.

3. The process for crystallizing an epoxy compound according to claim 1, wherein the temperature T° C. is from 30 to 60° C.

4. The process for crystallizing an epoxy compound according to claim 1, wherein the concentration of the epoxy compound in the solution containing a crude product of the epoxy compound is from 10 to 40% by weight and the temperature T¹ is a temperature which satisfies the following formula (III).
T¹<T−5 (III)

5. The process for crystallizing an epoxy compound according to claim 4, wherein the temperature T² is a temperature which satisfies the following formula (IV).
T−5<T²<T (IV)

6. The process for crystallizing an epoxy compound according to claim 4 or 5, wherein the temperature T³ is a temperature which satisfies the following formula (V):
−10<T³<T−5 (V)

7. The process for crystallizing an epoxy compound according to claim 4, wherein the temperature T¹ is from 10 to 30° C.

8. The process for crystallizing an epoxy compound according to claim 5, wherein the temperature T² is from 30 to 60° C.

9. The process for crystallizing an epoxy compound according to claim 6, wherein the temperature T³ is from −10 to 20° C.

10. The process for crystallizing an epoxy compound according to any one of claims 1 to 3, wherein Ar¹ and Ar³ independently denote a group represented by the following formula: 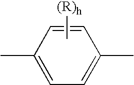
wherein R and h are as defined above.

11. The process for crystallizing an epoxy compound according to claim 10, wherein Ar¹ and Ar³ independently denote 1,4-phenylene group, 3-methyl-1,4-phenylene group or 3-isopropyl-1,4-phenylene group.

12. The process for crystallizing an epoxy compound according to any one of claims 1 to 3, wherein Ar² is a group represented by the following formula: 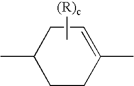
wherein R and c are as defined above.

13. The process for crystallizing an epoxy compound according to claim 12, wherein Ar² is 1-cyclohexene-1,4-diyl group.

14. The process for crystallizing an epoxy compound according to any one of claims 1 to 3, wherein Ar¹ and Ar³ independently denote a group represented by the following formula: 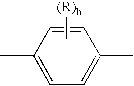
wherein R and h are as defined above, and Ar² denotes a group represented by the following formula: 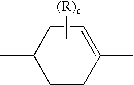
wherein R and c are as defined above.

15. The process for crystallizing an epoxy compound according to claim 14, wherein Ar¹ and Ar³ independently denote 1,4-phenylene group, 3-methyl-1,4-phenylene group or 3-isopropyl-1,4-phenylene group, and Ar² denotes 1-cyclohexene-1,4-diyl group.
